(19) United States
(12) Patent Application Publication
(10) Pub. No.: US 2006/0249147 A1
(43) Pub. Date: Nov. 9, 2006

(54) Aerosol dispenser

(75) Inventor: Mark Garon, Saint-Hyaclnthe (CA)

Correspondence Address: OGILVY RENAULT LLP, 1981 MCGILL COLLEGE AVENUE, SUITE 1600, MONTREAL, QC, H3A2Y3, CA

(73) Assignee: MULTI-VET LTD.

(21) Appl. No.: 11/399,606

(22) Filed: Apr. 7, 2006

Related U.S. Application Data
(60) Provisional application No. 60/669,391, filed on Apr. 8, 2005.

(51) Int. Cl. A61M 15/00; A61M 11/00

(52) U.S. Cl. 128/203.21; 128/200.23; 128/203.15; 128/203.19

(57) ABSTRACT

An aerosol delivery system dispenses an active substance using a propellant fluid. The delivery system comprises a sealed pressurizeable container having a dispensing nozzle. The propellant fluid is retained within the container and releasable therefrom in a gaseous phase via the dispensing nozzle when a release valve is actuated. The dispensing nozzle has a throat portion creating a venturi effect when the gaseous phase of the propellant fluid passes therethrough. A fluid containing reservoir is disposed within the container in fluid flow communication with the dispensing nozzle. The active substance is contained within the reservoir in isolation from the propellant fluid within the container. The venturi effect entrains the active substance through the dispensing nozzle for ejection therefrom when the release valve is actuated. A second reservoir may also be disposed within the container and provides a source of the gaseous propellant.

### TECHNICAL FIELD

The invention relates generally to a fluid delivery system, and more particularly, to an aerosol delivery system for dispensing an active substance using a pressurized gaseous propellant.

### BACKGROUND OF THE ART

Countless aerosol products are used in domestic households and in commercial and industrial applications. Aerosols are commonly used for the delivery of a variety of active substances, such as perfumes, air fresheners, repellents, cleaning products, paint, essential oils and countless other substances which may be dispensed in aerosolized form.

To dispense each of these products, an aerosol can is used which contains both a propellant and the active substance. Many different propellant fluids have been employed to dispense active substances. Typically, propellants are fluids which remain gaseous at normal atmospheric pressure but that assume a liquid phase when compresses at a few atmospheres of pressure. Aerosols generally work by mixing such a gas propellant with an active substance, which may or may not be mixed with each other within the container, and which are both expelled from the container by the pressure differential between the interior and exterior of the container.

It is well known that combining a solution of citric acid and sodium bicarbonate causes a reaction which produces CO2, however a residual salt by-product is also produced. Such a reaction has been used to generate CO2 for use as a propellant in aerosol dispensers, however the residual salt left over from the reaction tends to accumulate at the bottom of the container.

Thus, while carbon dioxide (CO2) has been used as a propellant in aerosol cans, several problems exist with such existing CO2 based systems. Particularly, the CO2 is never liquefied within the container (i.e. remains always in gaseous state) because the pressures necessary to do so would surpass the yield limits of the relatively thin walled container, which would therefore burst. Thus, the CO2 is not typically mixed with the active solution and only the liquid phase is expelled with the active substance. Accordingly, atomization of the CO2 propellant can only occur upon its exit from the container as a result of the selected actuator and spray nozzle. Further, if the active substance to be expelled by the aerosol container comes into contact with the accumulated residual salt, which is a by-product of the CO2 generation by mixing citric acid and sodium bicarbonate, contamination of the active substance can occur.

As such, there remains a need to provide an improved aerosol delivery system which will address at least these problems.

### SUMMARY OF THE INVENTION

It is therefore an object of this invention to provide an improved fluid delivery device having a gaseous propellant.

In one aspect, the present invention provides An aerosol delivery device for dispensing an active substance using a gaseous propellant, the delivery system comprising: a sealed pressurizeable canister defining a cavity therein for receiving said gaseous propellant, said cavity defining an internal volume; a dispensing nozzle disposed on said canister and a release valve in communication therewith, said release valve being in communication with said cavity and actuable to move between a closed position and an open position, said gaseous propellant being free to flow out of said cavity for ejection from said dispensing nozzle when said release valve is disposed in said open, said dispensing nozzle having a throat portion creating a venturi effect when said gaseous propellant passes therethrough; and a first reservoir for receiving the active substance therein disposed within said cavity of said canister and defining a first maximum internal volume less than the internal volume of said cavity, said first reservoir being in fluid flow communication with said throat portion of said dispensing nozzle such that said venturi effect entrains the active substance out of said first reservoir and through said dispensing nozzle for ejection therefrom when said release valve is disposed in the open position, said first reservoir being impermeable such as to isolate the active substance therewithin from the gaseous propellant within said cavity of said canister.

In accordance with another aspect of the present invention, there is provided a dispenser for dispensing at least one active substance in spray form comprising: a canister defining a cavity therein and having a dispensing nozzle in fluid flow communication with the cavity via a release valve, said cavity containing a pressurized gaseous propellant therein which is releasable from said dispensing nozzle when said release valve is opened; a first reservoir disposed within said cavity and containing said active substance therein, said first reservoir being impermeable such as to isolate the active substance therewithin from the gaseous propellant within said cavity and disposed in fluid flow communication with said dispensing nozzle, said first reservoir defining a first maximum internal volume; a second reservoir disposed within said cavity and providing a source of said gaseous propellant, said second reservoir defining a second maximum internal volume, said internal volume of said cavity being larger than said first and second maximum internal volumes combined; and a pressure regulator in pressure communication with at least said cavity such as to sense at least a first pressure of said gaseous propellant within said cavity, said pressure regulator allowing release of said gaseous propellant from within said second reservoir into said cavity when said first pressure is less than a reference pressure.

In accordance with another aspect of the present invention, there is also provided a method of dispensing at least one active substance in spray form from a portable canister, the method comprising: providing a pressurized gaseous propellant within a cavity of said canister; providing said active substance within an impermeable reservoir disposed within said cavity; opening a release valve of said canister to permit said pressurized gaseous propellant to flow out of a dispensing nozzle thereof; and generating a venturi effect within said dispensing nozzle to draw said active substance out of said reservoir and entrain it with said pressurized gaseous propellant through said dispensing nozzle for ejection therefrom.

There is further provided, in accordance with another aspect of the present invention, an aerosol delivery device for dispensing at least one active substance in spray form using an aerosol propellant, said aerosol delivery device comprising a canister defining an enclosed cavity therein in fluid flow communication with atmosphere outside said canister via a dispensing nozzle and a release valve, said dispensing nozzle having a venturi formed therein which generates a low pressure region at a throat thereof, a reservoir containing said active substance being disposed within said cavity in isolation from said aerosol propellant contained within said cavity outside said reservoir, said reservoir being in fluid flow communication with a portion of said dispensing nozzle adjacent said venturi, said aerosol propellant disposed within said cavity being releasable in gaseous form through said dispensing nozzle when said release valve is opened such that said venturi entrains said active substance into a flow of said aerosol propellant passing therethrough such as to form a mixture of said active substance and said aerosol propellant which is sprayed out of said dispensing nozzle.

Further details of these and other aspects of the present invention will be apparent from the detailed description and figures included below.

### DESCRIPTION OF THE DRAWINGS

Reference is now made to the accompanying figures depicting aspects of the present invention, in which:

FIG. 1 is a schematic cross-sectional view of an aerosol delivery system in accordance with the present invention.

### DETAILED DESCRIPTION OF THE PREFERRED EMBODIMENTS

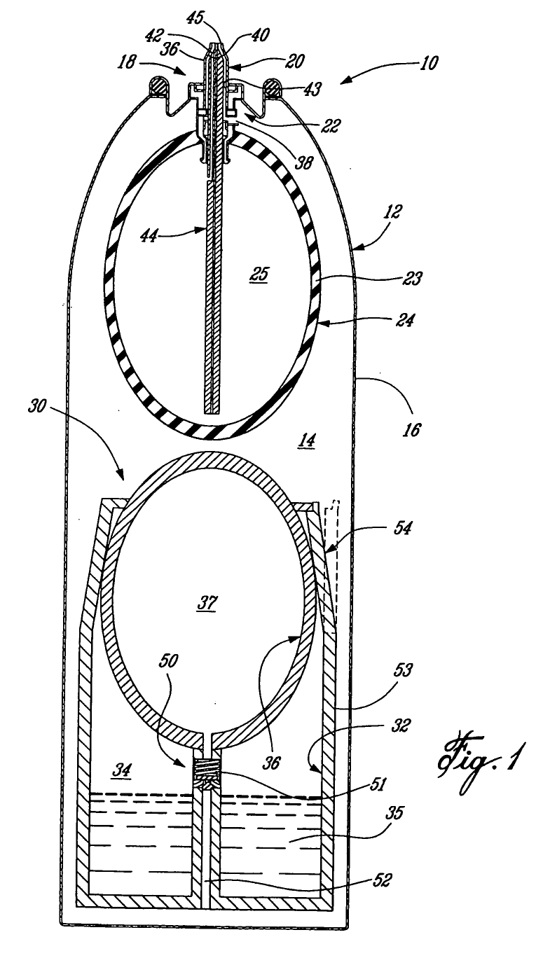

Referring to FIG. 1, a portable aerosol delivery system 10 includes generally a canister 12 defining a cavity 14 therewithin which is sealingly enclosed within the walls 16 of the canister 12. The canister is preferably an aerosol can which contains an aerosol propellant fluid within cavity 14. However, it is to be understood that other fluid propellants can also be used. A dispensing assembly 18 is preferably integrated into the aerosol canister 12 at the upper end thereof, however it remains possible that the dispensing assembly 18 is removably engaged to the canister. The dispensing assembly 18 includes a dispensing nozzle 20 and a release valve 22 which is actuable to open and close such that fluid is allowed to flow out of said canister 12 to the surrounding atmosphere, via the dispensing nozzle 20.

A first reservoir 24 is disposed within the cavity 14 of the canister 12, in fluid flow communication with the dispensing nozzle 20 of the dispensing assembly 18. The first reservoir 24 fits within the larger cavity 14 such that it may be preferably fully surrounded by propellant fluid disposed within the cavity 14 of the canister. The first reservoir therefore defines a maximum internal volume which is less than a total internal volume of the canister's main cavity 14. The first reservoir 24 contains an active substance, preferably in the form of a fluid, within the internal cavity 25 defined therein. The first reservoir 24 may be comprised of a deformable pouch, which is nonetheless impermeable such that the active substance therewithin is kept isolated from the surrounding propellant fluid within the canister's cavity 14. The active substance is preferably fluid-based and in a preferred embodiment comprises an effervescent fluid, however any other suitable active substance which can be dispensed by a propellant fluid from the canister may also be used. Although the aerosol delivery system 10 may be fully disposable, the canister 12 is preferably configured such that it can be opened, for example by removing the dispensing assembly 18, such that the first reservoir 24 containing the active substance therein may be replace when emptied. The first reservoir 24 may also be incorporated into the dispensing assembly 18, such that a replacement unit comprising both the dispensing assembly 18 and the active substance reservoir 24 may be purchased by the users as a single replacement part which replaces the existing corresponding components when the active substance within the first reservoir 24 has been consumed. In such an interchangeable system wherein the canister 12 is kept, the propellant reservoir assembly 30, which will be described in further detail below, may also be removed as a unit from the opened canister and replaced when necessary, such as when the reactants used to create the propellant gas are fully consumed.

A second, independent propellant reservoir assembly 30 may also be disposed within the cavity 14 of canister 12, separate from the first reservoir 24 containing the active substance. The propellant reservoir assembly 30, when provided, comprises at least two individual compartments or containers 32 and 36, which each respectively contain one of at least two reactants used to create, when combined such as to permit a chemical reaction therebetween, the gaseous propellant. Although any gaseous propellant may be used, for example pressurized air, inert gases, etc, in one embodiment the propellant used is gaseous carbon dioxide (CO2) which, once produced within the propellant reservoir assembly 30, is released therefrom and retained within the main cavity 14 of the canister 12. Preferably, the CO2 gas is produced by mixing sodium bicarbonate and citric acid. The citric acid is disposed within the upper cavity 37 defined within the first container 36, and the sodium bicarbonate 35 (which may be in solution) is disposed within the cavity 34 defined within the lower second container 32. A control valve 50 is disposed in fluid flow communication between these two reactant containers 36,32, and is also in pressure communication with the surrounding main cavity 14 of the canister 12 via a central passage 52. The control valve 50 may be a one-way valve as depicted in FIG. 1, or an alternate valve which can open and close to selectively permit the two reactants to mix together such as to form the gaseous propellant. The propellant reservoir 30 further includes pressure regulator 54, which provides selective communication between the inside of the reservoir 30 and the surrounding cavity 14 of the canister 12 such that gaseous propellant can flow out of the second reservoir and into the main cavity 14. The pressure regulator 54 includes, in the depicted embodiment, a displaceable portion 54 of an outer wall 53 of the propellant reservoir assembly 30 which is configured to pivot open when necessary, such that built up carbon dioxide gas within the propellant reservoir assembly 30, and more particularly within the second container 32, is released out into the main cavity 14 of the canister 12. However, alternate pressure regulators may be used, as will be apparent to one skilled in the art. For example, a one-way valve may be used in place of the displaceable wall portion. In another alternate embodiment, the pressure regulator 54 and the control valve 50 are integrated together such that a common structure performs both of their functions as described hereinabove.

In operation, when the pressure of the carbon dioxide gas within the cavity 14 of the canister 12 drops below a predetermined pressure level, the control valve 50, sensing the pressure drop via the passage 52, is forced open by the biasing member or spring 51 (the control valve 50 is biased in the closed position) and thus allowing citric acid within the cavity 37 of the first container 36 to flow into the cavity 34 of the first container 32 (or vice versa), where it is mixes with the solution of sodium bicarbonate 35 therein. Preferable, but not necessarily, the citric acid within cavity 37 flows downwards by gravity into the cavity 34 such as to mix with the sodium bicarbonate therein. Thus, carbon dioxide gas is created within this cavity 34. Alternately, however, the gaseous propellant (carbon dioxide in this case) may be formed within the other cavity, or within a third cavity within the second reservoir 30 separate from the reactant cavities 34,37. Once a sufficient quantity of carbon dioxide gas generated, and therefore once the internal pressure of gas within the propellant reservoir assembly 30 is greater than that outside the propellant reservoir assembly 30 within the cavity 14, the pressure regulator 54 opens thereby allowing the built up newly-generated carbon dioxide gas to be released into the main cavity 14. Once the pressures have equalized (i.e. the first pressure within the main cavity is no longer less than the second pressure within the second reservoir), the pressure regulator 54 re-closes to seal the propellant reservoir 30. Accordingly, the second/propellant reservoir 30 provides a source of gaseous propellant, which includes this propellant gas generating device which produces only propellant gas when needed, and is fully self correcting and self-generating until the two reactants, namely the sodium bicarbonate and the citric acid, are fully consumed. The pressure regulator 54 may alternately comprise other structures permitting release of the built up gas within the propellant reservoir 30 to escape out to the main cavity 14, such as for example a one-way or pressure relief valve operating autonomously or actuated by a suitable device, whether manually actuated by the user or remotely such as by an electronic control system.

When the propellant reactants are combined together within the reservoir 30, and the generated CO2 is subsequently released from the propellant reservoir assembly 30, the gaseous propellant is accumulated within the canister's cavity 14 as described above thus pressurizing the gaseous propellant such that when the release valve of the canister is opened the pressurized gaseous propellant will flow out through the dispensing nozzle. However, in the case when the gaseous propellant used is carbon dioxide, a by-product of the gaseous CO2 production is residual salt, which is either permitted to accumulate at the bottom of the propellant reservoir 30, or alternately at the bottom of the main cavity 14 of the canister itself, without risk of contaminating either the gaseous propellant within the cavity 14 or the active fluid separately contained within the first reservoir 24. Thus, as the active substance is always contained within the first reservoir 24, it remains isolated from the residual salt product left over from the reaction of the sodium bicarbonate and the citric acid to produce the gaseous CO2 used a propellant gas to entrain the active substance through the dispensing nozzle 20. The aerosol propellant and the active substance are thus both contained within the body of the canister 12, however they are both maintained isolated from one another until they mix together in the dispensing nozzle 20.

Although a second or propellant reservoir 30 may be provided within the cavity 14 of the canister, it is also possible to fill the cavity 14 with a pressurized gas (whether air, carbon dioxide, etc.), for use as the gaseous propellant within the cavity of the canister used to propel the active substance which remains contained within its impermeable and isolated reservoir 24 until mixing with the gaseous propellant immediately before ejected from the dispensing nozzle of the canister. In this embodiment, the whole dispensing device 10 may be discarded once all of the gaseous propellant or the active substance has been used up. Alternately still, once the gaseous propellant contained within the cavity 14 of the canister has been expelled, the cavity may be re-charged by new gaseous propellant, such as by pumping to pressurize air within the canister, injecting into the sealed canister a pre-pressurized gas such as nitrogen or carbon dioxide, or further still by injecting two reactants (such as citric acid and sodium bicarbonate) into the canister such that they combine therewithin to generate a self-pressurizing gas within the canister. An injection port, valve or other suitable one-way opening may be provided in the canister for such injection of either the gaseous propellant itself or fluid-based reactants which will mix together within the canister to generate the gaseous propellant.

The fluid-based active substance is, in one embodiment, passively drawn out of the first reservoir 24 by venturi effect of the stream of gaseous propellant flowing out of the dispensing nozzle 20, as described in further detail below. The active substance is passively entrained out of the reservoir 24 with the released gaseous propellant from within the cavity 14 of the canister 12. Alternately or in addition, it remains possible to actively feed the active substance out of the reservoir 24 and inject it into the stream of released aerosol propellant, such as by pumping, pressurization of the first reservoir 24, or otherwise forcing the fluid of the active substance out of its reservoir 24. In one embodiment, the active substance is both passive drawn out of its reservoir 24 by the venturi within the throat of the dispensing nozzle, as well as actively forced out by the pressure of the gaseous propellant within the cavity 14 of the canister which acts against the collapsible walls of the first reservoir 24, which in this embodiment is a deformable pouch.

The dispensing assembly 20 includes a dispensing nozzle 20 which is in fluid flow communication with both the first reservoir 24 and the cavity 14 of the canister 12 when a release valve 22 is actuated to open. The pressurized gaseous propellant, such as CO2 as described above, is then free to flow into the nozzle 20 via opening 38 and through the nozzle in annular passage 43 defined concentrically between a central tube 44 and the surrounding outer enclosure 36 of the nozzle 20. As the gaseous propellant passes through the annular passage 43 of the nozzle 20, it reaches a throat portion 40 near an outer end thereof which acts as a venturi, thus creating a low pressure venturi effect at the throat portion 40. An opening 42 is defined in the central tube 44 proximate this throat or venturi portion 40. Thus, the local depression near the opening 42 acts to passively draw the active substance from the cavity 25 of the reservoir 24 (which has a higher pressure than this depression) and up the tube 44. The active substance and the gaseous propellant then mix at this throat portion 40 before being ejected, in spray form or otherwise, from the tip 45 of the nozzle 20 due to the pressure and flow of propellant.

Thus, a passive system is provided for dispensing the active substance contained within the reservoir 24 in isolation, at least within the canister 12, from the gaseous propellant contained therein and the by-products of the generation of the gaseous propellant, such as the residual salt created during the formation of gaseous carbon dioxide from sodium bicarbonate and citric acid. However, as noted above, the displaceable wall portions 23 of the active substance reservoir 24 may be forced to collapsed inward towards each other by the pressure of the surrounding gaseous propellant within the larger cavity 14 of the canister, thereby acting to pressurize the active substance within the cavity 24 and thus help force the active substance out into the dispensing nozzle for mixing with the propellant before being ejected out of the nozzle tip 45.

Further, due to the isolation maintained between the active substance contained within the first reservoir 24 and the gaseous propellant, as well as the reactants and by-products produced during the production thereof, contamination of the active substance is prevented. In the dispenser 10 described herein, the gaseous propellant, whether CO2 or otherwise, is not mixed with the active solution until immediately before the sprayed ejected from the nozzle tip, and thus the active solution cannot become contaminated by the gaseous propellant or any constituent thereof, whether residual salt formed during the collection of carbon dioxide (which collects at the bottom of the reservoir 30 or at the bottom of the cavity 14) or otherwise. This enables the dispenser to take advantage of the fact that many more sprays are available which use gas phase of a propellant for dispensing, rather than for a liquid phase of a liquefiable propellant. In the present dispenser 10, when CO2 is used as the gaseous propellant the generated CO2 is accumulated inside the canister 12 and the residual salt solution is permitted to accumulate either at the bottom thereof or at the bottom of the propellant reservoir 30. The active ingredient is kept apart from this residual solution in the separate and isolated reservoir or pouch 24, and these never come into contact. This pouch 24 however, contrary to many existing systems, is disposed in direct contact with the air surrounding the dispenser when pressing on the actuator of the can, which operates the valve 22. The stream of propellant is then permitted to circulate over the outlet 42 of the active ingredient solution. The afore-mentioned venturi effect atomizes the liquid particles in the immediate environment, and the resulting spray is ejected from the dispensing nozzle of the dispenser.

Although the present invention has generally been described above with respect to an aerosol based propellant system, it is to be understood that other chemical propellants and/or pressurized or pressurizable gases, may be used as the propellant for dispensing the selected active substance fluid. Particularly, the canister 12 may contain merely compressed air for use as a propellant. In such an embodiment, the canister 12 may be provided with a pressurizing device for re-pressurizing the air contained therein, such as a manually or electronically operated pump for example. Alternately, pre-pressurized containers of air or another inexpensive and readily available gas may be used to re-fill the charged pressurized gas within the cavity 14 of the canister 12. Although the release valve 22 is preferably manually operable, it may alternately be open and closed remotely by a suitable actuation means.

The term active substance as used herein is intended to include all fluids and fluid-based substances having solid particles therein which may be dispensed in a sprayed aerosol form. As noted above, such an active substance is preferably a fluid, and may be any product which is dispensable in aerosol spray form, such as, for example only, perfumes, air fresheners, repellents, cleaning products, paint, and the like which is typically dispensed by an aerosol can. The active substance reservoir 24 may also contain fluids which are not typically dispensed in aerosol form, such as scented oils and the like.

The above description is meant to be exemplary only, and one skilled in the art will recognize that changes may be made to the embodiments described without department from the scope of the invention disclosed. Still other modifications which fall within the scope of the present invention will be apparent to those skilled in the art, in light of a review of this disclosure, and such modifications are intended to fall within the appended claims.

1. An aerosol delivery device for dispensing an active substance using a gaseous propellant, the delivery system comprising:
a sealed pressurizeable canister defining a cavity therein for receiving said gaseous propellant, said cavity defining an internal volume;
a dispensing nozzle disposed on said canister and a release valve in communication therewith, said release valve being in communication with said cavity and actuable to move between a closed position and an open position, said gaseous propellant being free to flow out of said cavity for ejection from said dispensing nozzle when said release valve is disposed in said open, said dispensing nozzle having a throat portion creating a venturi effect when said gaseous propellant passes therethrough; and
a first reservoir for receiving the active substance therein disposed within said cavity of said canister and defining a first maximum internal volume less than the internal volume of said cavity, said first reservoir being in fluid flow communication with said throat portion of said dispensing nozzle such that said venturi effect entrains the active substance out of said first reservoir and through said dispensing nozzle for ejection therefrom when said release valve is disposed in the open position, said first reservoir being impermeable such as to isolate the active substance therewithin from the gaseous propellant within said cavity of said canister.

2. The aerosol delivery device as defined in claim 1, wherein a second reservoir is disposed within said cavity of said canister from which said gaseous propellant is provided, said second reservoir defines a second maximum internal volume, said first and second maximum internal volumes being together less than the internal volume of said cavity.

3. The aerosol delivery device as defined in claim 2, wherein said second reservoir includes a pressure regulator permitting flow of gas within said second reservoir out to said cavity of said canister when said pressure regulator is disposed in an open position.

4. The aerosol delivery device as defined in claim 3, wherein said second reservoir includes a gas generating device having at least two reactant compartments interconnected by a control valve displaceable between a closed position and an open position, said control valve allowing fluid flow communication between said internal compartments when disposed in said open position such as to permit mixing of reactants which generate said gaseous propellant.

5. The aerosol delivery device as defined in claim 4, wherein said control valve is disposed in pressure communication with said cavity such as to sense a first pressure of the gaseous propellant therewithin.

6. The aerosol delivery device as defined in claim 5, wherein said control valve is self regulating, said valve being autonomously displaceable from said closed position to said open position in response to a sensed drop in said first pressure.

7. The aerosol delivery device as defined in claim 4, wherein said control valve and said pressure regulator are biased in their respective closed positions.

8. The aerosol delivery device as defined in claim 4, wherein said pressure regulator is disposed within a wall portion separating one of said reactant compartments and said cavity of said canister.

9. The aerosol delivery device as defined in claim 3, wherein said pressure regulator is self regulating, being displaceable to said open position when a pressure within said cavity of said canister drops below a predetermined level.

10. The aerosol delivery device as defined in claim 9, wherein said predetermined level corresponds to an internal pressure within said second reservoir.

11. The aerosol delivery device as defined in claim 10, wherein said pressure regulator includes at least one of a pivotable wall section and a one-way valve.

12. The aerosol delivery device as defined in claim 1, wherein said first reservoir is a deformable pouch.

13. The aerosol delivery device as defined in claim 1, wherein said active substance includes an effervescent fluid.

14. The aerosol delivery device as defined in claim 1, wherein said gaseous propellant is carbon dioxide.

15. A dispenser for dispensing at least one active substance in spray form comprising:
a canister defining a cavity therein and having a dispensing nozzle in fluid flow communication with the cavity via a release valve, said cavity containing a pressurized gaseous propellant therein which is releasable from said dispensing nozzle when said release valve is opened;
a first reservoir disposed within said cavity and containing said active substance therein, said first reservoir being impermeable such as to isolate the active substance therewithin from the gaseous propellant within said cavity and disposed in fluid flow communication with said dispensing nozzle, said first reservoir defining a first maximum internal volume;
a second reservoir disposed within said cavity and providing a source of said gaseous propellant, said second reservoir defining a second maximum internal volume,. said internal volume of said cavity being larger than said first and second maximum internal volumes combined; and
a pressure regulator in pressure communication with at least said cavity such as to sense at least a first pressure of said gaseous propellant within said cavity, said pressure regulator allowing release of said gaseous propellant from within said second reservoir into said cavity when said first pressure is less than a reference pressure.

16. The dispenser as defined in claim 15, wherein said pressure regulator senses a second pressure of said gaseous propellant within said second reservoir, said second pressure being said reference pressure.

17. The dispenser as defined in claim 16, wherein said pressure regulator is autonomously self regulating.

18. The dispenser as defined in claim 17, wherein said pressure regulator includes at least one of a displaceable wall portion and a pressure relief valve disposed between said second reservoir and said cavity, said at least one of said displaceable wall portion and said pressure relief valve being displaceable between an open position allowing fluid flow communication between said second reservoir and said cavity and a closed position sealing separating said second reservoir and said cavity.

19. The dispenser as defined in claim 18, wherein said at least one of said displaceable wall portion and said pressure relief valve is biased in said closed position.

20. The dispenser as defined in claim 15, wherein said source of said gaseous propellant includes a gas generating device, said gas generating device selectively generating said gaseous propellant within said second reservoir.

21. The dispenser as defined in claim 20, wherein said gas generating device includes a control valve in pressure communication with said cavity such as to sense said first pressure and with said second reservoir such as to sense a second pressure therewithin, said control valve being displaceable between an open position wherein said gaseous propellant is generated and a closed position wherein generation of said gaseous propellant is ceased.

22. The dispenser as defined in claim 21, wherein said open position is defined by said first pressure being less than said second pressure and said closed position is defined by said first pressure being equal to or greater than said second pressure.

23. The dispenser as defined in claim 21, wherein said gas generating device includes at least two compartments within said second reservoir each containing a reactant and being interconnected by said control valve, said control valve permitting fluid flow communication between said at least two compartments when disposed in said open position such that said reactants combine to chemically react and generate said gaseous propellant within said second reservoir.

24. The dispenser as defined in claim 15, wherein said gaseous propellant includes carbon dioxide.

25. The dispenser as defined in claim 23, wherein said gaseous propellant includes carbon dioxide and said reactants include sodium bicarbonate and citric acid.

26. A method of dispensing at least one active substance in spray form from a portable canister, the method comprising:
providing a pressurized gaseous propellant within a cavity of said canister;
providing said active substance within an impermeable reservoir disposed within said cavity;
opening a release valve of said canister to permit said pressurized gaseous propellant to flow out of a dispensing nozzle thereof; and
generating a venturi effect within said dispensing nozzle to draw said active substance out of said reservoir and entrain it with said pressurized gaseous propellant through said dispensing nozzle for ejection therefrom.

27. The method as defined in claim 20, further comprising generating the gaseous propellant within said cavity by mixing at least two reactants.

28. The method as defined in claim 27, further comprising providing a second reservoir within said cavity of said canister, and performing said step of generating within said second reservoir.

29. An aerosol delivery device for dispensing at least one active substance in spray form using an aerosol propellant, said aerosol delivery device comprising a canister defining an enclosed cavity therein in fluid flow communication with atmosphere outside said canister via a dispensing nozzle and a release valve, said dispensing nozzle having a venturi formed therein which generates a low pressure region at a throat thereof, a reservoir containing said active substance being disposed within said cavity in isolation from said aerosol propellant contained within said cavity outside said reservoir, said reservoir being in fluid flow communication with a portion of said dispensing nozzle adjacent said venturi, said aerosol propellant disposed within said cavity being releasable in gaseous form through said dispensing nozzle when said release valve is opened such that said venturi entrains said active substance into a flow of said aerosol propellant passing therethrough such as to form a mixture of said active substance and said aerosol propellant which is sprayed out of said dispensing nozzle.
